(19) United States
(12) Patent Application Publication
(10) Pub. No.: US 2026/0158007 A1
(43) Pub. Date: Jun. 11, 2026

(54) INHIBITION OF BACH1 FOR PULMONARY HYPERTENSION THERAPY

(71) Applicant: Ann and Robert H. Lurie Children's Hospital of Chicago, Chicago, IL (US)

(72) Inventor: Youyang Zhao, Chicago, IL (US)

(21) Appl. No.: 19/413,434

(22) Filed: Dec. 9, 2025

Related U.S. Application Data
(60) Provisional application No. 63/729,678, filed on Dec. 9, 2024.

(51) Int. Cl. A61K 31/428; A61K 31/423; A61P 9/12

(52) U.S. Cl. CPC A61K 31/428; A61K 31/423; A61P 9/12

(57) ABSTRACT

The present invention provides methods of reducing or inhibiting pulmonary vascular remodeling and methods of treating pulmonary hypertension by administering an inhibitor of BACH1.

### CROSS-REFERENCE TO RELATED APPLICATIONS

This application claims priority to U.S. Provisional Application No. 63/729,678 filed on Dec. 9, 2024. The contents of which is incorporated by reference in its entirety.

### STATEMENT REGARDING FEDERALLY SPONSORED RESEARCH

This invention was made with government support under grant numbers R01HL133951 and R01HL162299 awarded by the National Institutes of Health. The government has certain rights in the invention.

### REFERENCE TO AN ELECTRONIC SEQUENCE LISTING

The contents of the electronic sequence listing (70258102729.xml; Size: 2,704 bytes; and Date of Creation: Dec. 9, 2025) is herein incorporated by reference in its entirety.

### BACKGROUND

Pulmonary arterial hypertension (PAH) is characterized by progressive increases of pulmonary vascular resistance and vascular remodeling, which without treatment leads to right heart failure and premature death. As the molecular mechanisms responsible for pulmonary vascular remodeling remain elusive, there are limited options available for the prevention and treatment of progressive PAH. Therapies targeting abnormalities in the prostacyclin, nitric oxide, and endothelin pathways result in only modest improvement in morbidity and mortality. PAH types with different etiologies share histopathologic features including obliterative intima thickening and complex plexiform lesions. In view of the foregoing, it would be desirable to delineate the signaling pathway(s) mediating progressive pulmonary vascular remodeling, and thereby identify novel therapeutic strategies to inhibit or potentially reverse pulmonary vascular remodeling for PAH treatment.

### SUMMARY

This disclosure describes methods of treating pulmonary hypertension and the fundamental role of endothelial BACH1 in mediating pulmonary vascular remodeling and PAH development. Unlike existing therapies that target prostacyclin, nitric oxide, and endothelin pathways and result in only modest improvements in morbidity and mortality, the present technology provides a novel therapeutic approach by specifically targeting BACH1. This approach overcomes the limitations of current treatments, which have limited efficacy and leave patients with progressive disease leading to right heart failure and premature death. The disclosed methods demonstrate marked reduction in pulmonary vascular resistance and vascular remodeling through inhibition of BACH1, which is believed to act via downstream transcription factors HIF-2α and PPARγ. This targeted mechanism provides superior therapeutic outcomes compared to conventional treatments, offering effective prevention and treatment of progressive PAH where current therapeutic options remain inadequate.

One aspect of the present disclosure provides a method of treating pulmonary hypertension in a subject in need thereof, the method comprising administering an inhibitor of BACH1 to the subject. In some embodiments, the pulmonary hypertension is pulmonary arterial hypertension. In some embodiments, the BACH1 inhibitor is selected from the group consisting of antibodies, small molecule therapeutic agents, peptides, miRNAs, siRNAs, oligonucleotides, genome editors, protein degraders, cytokines, agonists and combinations thereof. In some embodiments, the BACH1 inhibitor comprises ASP8731, HPP-4382, HPP971, HPPA, HPPB, HPPC, HPPD, HPPE, TBE31, TBE53, TBE56, cannabiquinone, cannabidiol, IMM004, hemin, CDDO-TFEA, CDDO-Me, BIO33, 1-piperazineethanol, α-[(1,3-benzodioxol-5-yloxy) methyl]-4-(2-methoxyphenyl) (M2), and combinations thereof. In some embodiments, the method decreases vascular resistance. In some embodiments, BACH1 inhibitor decreases BACH1 expression and/or function in endothelial cells.

Another aspect of the present disclosure provides a method of inhibiting or reducing pulmonary vascular remodeling in a subject in need. In some embodiments, the method comprises administering an inhibitor of BACH1 to the subject. In some embodiments, the subject is diagnosed with or suspected of having pulmonary hypertension. In some embodiments, the BACH1 inhibitor is selected from the group consisting of antibodies, small molecule therapeutic agents, peptides, miRNAs, siRNAs, oligonucleotides, genome editors, protein degraders, cytokines, agonists and combinations thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present technology can be better understood by reference to the following drawings. The drawings are merely exemplary to illustrate certain features that may be used singularly or in combination with other features and the present technology should not be limited to the embodiments shown.

FIG. 1A-1B. Immunostaining demonstrating diminished Bach1 expression in Bach^{Δ}^{_{EC}} rat lung ECs but not in WT lung ECs. (A) Diagram presentation of EndoNP1 nanoparticle delivery of the CRISPR plasmid DNA to rats. (B) Representative micrographs of anti-Bach1 immunostaining (green) in rat lung cryosections. ECs were immunostained with anti-CD31 (red) and nuclei were counterstained with DAPI (blue). Arrows point to Bach1 expression in non-ECs.

FIG. 2A-2B. Marked inhibition of PAH in MCT-CKO rats compared to WT-MCT rats. (A) RVSP measurement. (B) RV/LV+S ratio. CTL (control)=WT rats without MCT challenge. ****P<0.0001. One-way ANOVA with Tukey's correction for multiple group comparison.

FIG. 3A-3G. Echocardiography demonstrating normal RV and PA functions in MCT-CKO rats in contrast to MCT-WT rats. (A) Representative micrographs of TAPSE (Tricuspid Annular Plane Systolic Excursion) measurement. (B-G) Bar graph presentations of TPASE values, RVFAC (Right Ventricular Fractional Area Change), RV diastolic chamber size, RV diastolic wall thickness, Cardiac Output, and PAAT/ET (Pulmonary Artery Acceleration Time/RV Ejection Time) ratio. *P<0.05; **P<0.01; ****P<0.0001. n.s=not significant. One-way ANOVA with Tukey's correction.

FIG. 4A-4C. Reduced pulmonary vascular remodeling in MCT-CKO rats. (A) Representative micrographs of pentachrome staining of rat lung sections. Arrows point to vessels. Br, bronchiole; V, vessel. (B) Quantification of pulmonary vessel media wall thickness. Fractional MWT=media wall thickness/diameter. (C) Quantification of muscularized distal pulmonary vessels. The average number of α-SMA-positive distal pulmonary vessels (d≤50 μm) of 20× fields of each section was used for each rat. Scale bars, 50 μm. **P<0.01; ****P<0.001. One-way ANOVA with Tukey's correction.

FIG. 5A-5B. Reduced PASMC proliferation in MCT-CKO rat lungs compared to MCT-WT rat lungs. (A) Representative micrographs of anti-α-SMA (SMA, green) and anti-Ki67 (red) immunostaining of rat lung cryosections. Nuclei were counterstained with DAPI (blue). Arrows point to proliferating SMCs. Scale bar, 50 μm. (B) Quantification of PASMC proliferation. *P<0.05. Mann-Whitney Utest.

FIG. 6A-6D. Marked induction of BACH1 expression in lung ECs of IPAH patients and also in normal lung ECs by PAH-causing stimuli. (A) Quantitative RT-PCR analysis of BACH1 and BACH2 expression in primary cultures of pulmonary arterial ECs isolated from unused donor lungs (normal) and IPAH lungs. (B) representative micrographs of immunostaining demonstrating prominent BACH1 expression in ECs of pulmonary vascular lesions of IPAH patients but not in normal donor lungs. Formalin-fixed lung sections were immunostained with anti-BACH1 (red) and anti-vWF (green). Nuclei were counterstained with DAPI (blue). V, vessel. (C, D) Quantitative RT-PCR analysis demonstrating marked induction of BACH1 expression in primary cultures of human pulmonary artery ECs isolated from unused donor lungs by PDGF-BB (PDGF) treatment (C) or in human lung microvascular ECs isolated from normal donor lungs by hypoxia (Hx) exposure (D). Nx=normoxia. n.s., not significant. ***P<0.001; ****P<0.0001. Unpaired 2-tailed t-test.

FIG. 7. RNAseq analysis identifying HIF-2α and PPARy as top transcription factors mediating differential expression genes (DEGs). RNA samples were extracted from lung tissues of basal-WT, MCT-WT and MCT-CKO lungs with Trizol reagent followed by Qiagen RNAeasy purification including DNase I digestion for RNAseq analysis.

FIG. 8. Quantitative RT-PCR analysis demonstrating increased Pparg mRNA expression in MCT-CKO lugs compared to MCT-WT lungs. *P<0.05. One-way ANOVA with Tukey's correction.

FIG. 9. Immunostaining demonstrating prominent HIF-2α protein levels in ECs of MCT-WT rat lungs but neither basal-WT nor MCT-CKO lungs. Cryosections of rat lungs were immunostained with anti-HIF-2α antibody (red). ECs were immunostained with anti-vWF (green). Nuclei were counterstained with DAPI (blue). Arrows point to ECs expressing abundant HIF-2α protein. V, vessel; Br, bronchiole.

FIG. 10A-10E. Overexpression of HIF-2α mutant and inhibition of PPARγ reversed the protective phenotype in MCT-CKO rats, supporting HIF-2α and PPARγ as the targets of endothelial Bach1 responsible for PAH development. After 3-weeks MCT challenge, the rats were subject to hemodynamic measurement and echocardiography. Measurements of (A) RVSP; (B) RV/LV+S Ratio; (C) TAPSE; (D) RVFAC; and (E) PA AT/ET Ratio are shown. Vec=empty vector DNA without HIF-2α; HIF-2α=HIF-2α mutant; Pi=PPARγ inhibitor.

FIG. 11A-11C. Overexpressing BACH1-ECs induced PASMC hyperproliferation in co-culture system. (A) Model of co-culture; (B) 24 h after co-culture, PASMCs were fixed for immunostaining with anti-Edu (purple). Nuclei were counterstained with DAPI (blue). **P<0.01. unpaired 2-tailed t test and (C) quantified.

FIG. 12A-12C. Treatment with BACH1 inhibitor ASP8731 inhibited PH in MCT rats. (A) Chemical structure of ASP8731. (B, C) 14 days after MCT challenge, the rats were treated with either vehicle or ASP8731 (25 mg/kg body weight, gavage, daily). 14 days later, RVSP (B) and RV hypertrophy (C) were assessed.

FIG. 13A-13C. Inhibited PH in HPPE-treated rats. (A) Chemical structure of HPPE. (B, C) 10 days after MCT challenge, the rats were treated with either vehicle or HPPE (50 mg/kg body weight, i.p. daily, every 4 days)-100 mg/kg. At 28 days after MCT challenge, RVSP (B) and RV hypertrophy (C) were determined.

### DETAILED DESCRIPTION

The transcription factor BTB and CNC homology 1 (BACH1) belongs to the Cap ‘n’ Collar (CNC) b-Zip family of proteins. There is no information about the role of BACH1 in regulating pulmonary vascular remodeling and pulmonary arterial hypertension development. It was observed that BACH1 but not BACH2 expression was markedly increased in endothelial cells (ECs) in idiopathic PAH lungs compared to normal lungs, and in cultured human pulmonary arterial ECs treated with PDGF or hypoxia. Rats with endothelial cell-specific knockout of BACH1 via nanoparticle delivery of the CRISPR system exhibited markedly reduced pulmonary vascular remodeling and PAH in response to monocrotaline challenge. RNA sequencing analysis and in vivo rescue study have identified HIF-2α and PPARgamma as the downstream transcription factors responsible for Bach1-mediated pulmonary vascular remodeling and PAH. In vivo inhibition of PPARgamma and activation of HIF-2α together reversed the protective phenotype seen in Bach1 EC-specific knockout rats. Data disclosed herein have for the first time demonstrated the fundamental role of endothelial BACH1 in mediating pulmonary vascular remodeling and PAH development. Based on the clear clinical relevance of these novel findings, inhibition of BACH1, especially endothelial BACH1 by small molecule inhibitor, siRNA, antisense, antibody, dominant negative BACH1, genome editing by CRISPR, Prime editing, Base editing and other ways disrupting BACH1 expression, subcellular localization, or inhibiting its function is a novel and effective PAH therapy.

### Methods:

One aspect of the present disclosure provides methods of treating pulmonary hypertension in a subject in need thereof.

A “subject in need thereof” as utilized herein may refer to a subject in need of treatment for a disease or disorder associated with pulmonary hypertension. The term “subject” may be used interchangeably with the terms “individual” and “patient” and includes human and non-human mammalian subjects.

As used herein, “subject” or “patient” refers to both mammals and non-mammals. “Mammals” include any member of the class Mammalia, such as humans, non-human primates (e.g., chimpanzees, other apes and monkey species), farm animals (e.g., cattle, horses, sheep, goats, and swine), domestic animals (e.g., rabbits, dogs, and cats), and laboratory animals (e.g., rats, mice, and guinea pigs). The term “subject” does not denote a particular age or sex. In one embodiment, the subject is a human. In some embodiments, the subject is diagnosed with or is suspected of having pulmonary hypertension.

In pulmonary hypertension, the arteries in the lungs can narrow and then the blood does not flow as well as it should, resulting in less oxygen in the blood. Pulmonary hypertension can develop on its own or can be caused by another condition. Conditions which can cause pulmonary hypertension include, but are not limited to heart conditions such as heart failure, congenital heart defects, coronary artery disease, and heart valve disease; lung conditions such as chronic obstructive pulmonary disease, interstitial lung disease, emphysema, and sleep apnea; and blood vessel conditions such as blood clots in the lungs (pulmonary embolism), tumors that block the pulmonary artery. Genetics and medications can also cause pulmonary hypertension. In some embodiments, the pulmonary hypertension is pulmonary arterial hypertension (PAH). PAH is a progressive disease of the lung vascular system, primarily affecting the small pulmonary arterioles. A combination of endothelial dysfunction and increased contractility of small pulmonary arteries, proliferation and remodeling of endothelial and smooth muscle cells, and in situ thrombosis leads to progressive narrowing of the blood vessels. This results in a progressive resistance to blood flow and an increase in pulmonary artery pressures.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “treat,” “treatment,” and “treating” refer to reducing the amount or severity of a particular condition, disease state (e.g., cancers), or symptoms thereof, in a subject presently experiencing or afflicted with the condition or disease state. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process. Specifically, treatment may result in the improvement of blood pressure measurements, pulmonary function, heart function, vascular remodeling and or vascular resistance. As used herein, the term “treatment” is not necessarily meant to imply cure.

As used herein, the term “administering” an agent, such as a therapeutic entity or composition described herein to a subject or cell, is intended to refer to dispensing, delivering or applying the substance to the intended target. In terms of the therapeutic agent composition, such as a BACH1 inhibitor, the term “administering” is intended to refer to contacting or dispensing, delivering or applying the therapeutic agent to a subject by any suitable route for delivery of the therapeutic agent to the desired location in the subject, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route. A BACH1 inhibitor described herein may be administered one or more times, and in combination with other treatments or alone. In some embodiments more than one BACH1 inhibitor may be used.

Means of detecting and measuring pulmonary hypertension are known in the art, and include, but are not limited to echocardiography, right heart catheterization, electrocardiogram, chest x-ray, blood tests, nuclear lung scan, and cardiopulmonary exercise testing. Treating pulmonary hypertension may include detecting an improvement of blood pressure measurements, pulmonary function, heart function, vascular remodeling and or vascular resistance as well as 6-minutes walk distance.

In some embodiments, a method of treating pulmonary hypertension comprises administering an inhibitor of BACH1 to the subject. The transcription factor BTB and CNC homology 1 (BACH1) belongs to the Cap ‘n’ Collar (CNC) b-Zip family of proteins.

As used herein, the term “BACH1 inhibitor” refers to any compound or molecule that is capable of inhibiting the action of, and or reducing the expression and or function of BACH1. In some embodiments, the BACH1 inhibitor may comprise an antibody, small molecule therapeutic agents, a miRNA, siRNA, oligonucleotides, cytokines, agonists, a dominant negative BACH1, a BACH1 protein degrader, a genome editor, and combination thereof Small molecule therapeutic agent generally refers to compound of low molecular weight, such as less than 1000 Da, 900 Da, 800 Da, 700 Da, 600 Da, or 500 Da, and small size, such as a size on the order of 1 nm or less, than may regulate a biological process. Small molecule therapeutic agents may include compounds capable of passive diffusion across a cell membrane as well as those transported to intracellular locations via a drug delivery vehicle, ion channels, cellular transport mechanism, or any other suitable means. Suitable examples of BACH1 inhibitors include, but are not limited to ASP8731 (CAS No. 2488255-42-9), HPPE (CAS No. 1325721-55-8), HPP-4382, HPPA, HPPB, HPPC, HPPD, HPP971, TBE31 (CAS No. 936475-62-6), TBE53, TBE56 (CAS No. 1459836-79-3), Cannabidiol (CAS No. 13956-29-1), cannabiquinone (CAS No. 137252-25-6), IMM004 (IMMvention Therapeutix, Inc.), hemin (CAS No. 16009-13-5), CDDO-TFEA (CAS No. 932730-52-4), CDDO-Me (CAS No. 218600-53-4), BI033 (CAS No. 880810-80-0), 1-piperazineethanol (CAS No. 103-76-4), M2 (CAS No. 380192-64-3) and analogs thereof In some embodiments, the inhibitor comprises a HPPE analog. Genome editors comprise nucleases, such as Cas nuclease, transcription activator-like effect based nuclease (TALEN), zinc finger nuclease or meganuclease; base editor, a prime editor and a transposase. Oligonucleotides used to inhibit BACH1 may comprise those used in genomic editing such as prime-editing, base editing, CRISPR editing or non-coding RNA such as miRNA or siRNA or antisense. For example, gRNA (SEQ ID NOs: 1 and 2) was used to generate a BACH1 knockout. In some embodiments, species specific oligonucleotides, including gRNAs may be used to knock out BACH1.

In some embodiments, BACH1 inhibitors may be combined with other known therapeutics or treatments for pulmonary hypertension or vascular remodeling. By way of example, and not limitation, common treatments may include calcium channel blockers, diuretics, oxygen therapy, pulmonary vasodilators, sotatercept, anticoagulants, surgery and lifestyle changes.

Pulmonary hypertension (PH) occurs when blood pressure in the lungs is abnormally high. Pulmonary vascular remodeling results in high pulmonary vascular resistance. Vascular remodeling refers to alterations in the structure of resistance vessels contributing to elevated vascular resistance.

In some embodiments, the BACH1 inhibitor deceases vascular resistance. In some embodiments the BACH1 inhibitor decreases vascular remodeling. In some embodiments, the vascular resistance and vascular remodeling are pulmonary vascular resistance and pulmonary vascular remodeling. Means of detecting and measuring vascular resistance and remodeling are known in the art. For example, vascular resistance can be measured using a mathematical formula by calculating the mean arterial pressure from blood pressure readings, central venous pressure and cardiac output, or by using echocardiograph. Similarly, vascular remodeling can be detected and or measured by calculating a percentage using the maximal outer diameter of an artery and the diameter of an artery at a site of a plaque. Vascular remodeling can also be detected and or measured by imaging, such as ultrasound, myograph, serial IVUS and photo acoustics. Inward remodeling is when the vessel size decreases, while outward remodeling is when the vessel size increases. As described herein, loss of BACH1 or inhibition of BACH1 reduces right ventricular systolic pressure (RVSP) and RV/LV+S ratio.

As used herein, the term “decrease” or the related terms “decreased,” “reduce” or “reduced” refers to a statistically significant decrease. The term “increase” or the related terms “increased,” “upregulate” or “upregulated” refers to a statistically significant increase. For the avoidance of doubt, the terms generally refer to at least a 10% decrease or increase in a given parameter, and can encompass at least a 20% decrease or increase, 30% decrease or increase, 40% decrease or increase, 50% decrease or increase, 60% decrease or increase, 70% decrease or increase, 80% decrease or increase, 90% decrease or increase, or a 100% decrease or increase or even more than 100% increase (i.e., the measured parameter is at zero). An increase or decrease may be represented as an absolute a value, or a relative value such as a ratio, or percentage. In some embodiments, decreasing vascular resistance or decreased vascular remodeling may refer to one or more measurements of vascular resistance or vascular remodeling.

BACH1 is a transcription factor with multiple downstream targets, including Foxf1, HIF2α, PPARgamma, Foxl1, Tcf21, NRld1, Tbx4, Snai1, Erg and Tbx2. Inhibition of BACH1 may alter one or more of these downstream targets. In some embodiments, a BACH1 inhibitor may decrease HIF-2α and or increase PPARγ.

In some embodiments, expression or amount is measured, such as expression of BACH1, HIF2α, or PPARγ. Expression may be measured by any means known in the art, including, but not limited to Western blot, ELISA, immunohistochemistry, immunofluorescence, spectrophotometry, colorimetric or fluorometric assay, quantitative PCR, flow cytometry, digital PCR, or sequencing techniques.

Another aspect of the present disclosure provides, a method of inhibiting or reducing vascular remodeling in a subject in need is provided. The method comprising administering a BACH1 inhibitor to the subject. Vascular remodeling refers to structural changes in the blood vessels, including vessel wall structure, and can lead to vascular resistance.

Another aspect of the present disclosure provides a method of inhibiting or reducing pulmonary vascular remodeling in a subject in need, the method comprising administering an inhibitor of BACH1 to the subject, wherein the subject is diagnosed with or suspected of having pulmonary hypertension. Pulmonary vascular remodeling refers to structural changes in the pulmonary arteries and distal pulmonary vessels, including pulmonary vessel wall structural changes and can lead to increased pulmonary vascular resistance and pulmonary hypertension.

An aspect of the technology encompasses pharmaceutical compositions comprising a BACH1 inhibitor together with one or more pharmaceutically acceptable carriers, excipients, or diluents. Such compositions may be formulated for any suitable route of administration, such as oral, parenteral, intravenous, intramuscular, subcutaneous, intradermal, intrathecal, buccal, transdermal, topical, intranasal, respiratory tract, or other suitable routes of administration. The pharmaceutically acceptable carrier may be selected based on the intended route of administration and standard pharmaceutical practice. In some embodiments, the pharmaceutical composition may be formulated as a tablet, capsule, solution, suspension, emulsion, aerosol, powder, or other suitable dosage form. The pharmaceutical composition may further comprise additional therapeutic agents for the treatment of pulmonary hypertension or vascular remodeling.

Another aspect of the technology encompasses a BACH1 inhibitor or a pharmaceutical composition comprising said BACH1 inhibitor, for use in the treatment of pulmonary hypertension in a subject. The BACH1 inhibitor or pharmaceutical composition may be used in the treatment of pulmonary arterial hypertension. The BACH1 inhibitor or pharmaceutical composition may also be used for inhibiting or reducing vascular remodeling in a subject. The BACH1 inhibitor or pharmaceutical composition may be used in inhibiting or reducing pulmonary vascular remodeling in a subject diagnosed with or suspected of having pulmonary hypertension.

Another aspect of the technology encompasses the use of a BACH1 inhibitor for the manufacture of a medicament. Suitably, may be for the treatment of pulmonary hypertension, treatment of pulmonary arterial hypertension, inhibiting or reducing vascular remodeling, or inhibiting or reducing pulmonary vascular remodeling in a subject diagnosed with or suspected of having pulmonary hypertension.

### Additional Definitions

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps.

All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

“Percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a, nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Snaith and Waterman, (1981) Adv. Appl. Math. 2.482, by the homology alignment algorithm of Needleman and Wunsch, (1970) J. Mol. Biol. 48443, by the search for similarity method of Pearson and Lipman, (1988) Proc. Natl. Acad. Sci. USA 85:2444, by computerized implementations of these algorithms (GAP, BESTFIT, FASTA, and TFASTA), or by visual inspection (see generally, Current Protocols in Molecular Biology, F. M. Ausubel et al., eds., Current Protocols, Greene Publishing Associates, Inc. and John Wiley & Sons, Inc., (1995 Supplement)).

Examples of algorithms that are suitable for determining percent sequence identity and sequence similarity are the BLAST and BLAST 2.0 algorithms, which are described in Altschul et al., (1990), J. Mol. Biol. 215: 403-410 and Altschul et al., (1977) Nucleic Acids Res. 3389-3402, respectively. Software for performing BLAST analyses is publicly available through the National Center for Biotechnology Information website.

In those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The following examples are meant only to be illustrative and are not meant as limitations on the scope of the invention or of the appended claims.

### EXAMPLES

The following Examples are illustrative and should not be interpreted to limit the scope of the claimed subject matter.

### Example 1: Inhibition of BACH1 for Pulmonary Arterial Hypertension Therapy

Pulmonary arterial hypertension (PAH) is characterized by progressive increases of pulmonary vascular resistance and vascular remodeling, which without treatment leads to right heart failure and premature death¹⁻³. As the molecular mechanisms responsible for pulmonary vascular remodeling remain elusive, there are limited options available for the prevention and treatment of PAH. It is important to delineate the signaling pathway(s) mediating progressive pulmonary vascular remodeling, and thereby identify novel therapeutic approaches to effectively treat PAH. The transcription factor BTB and CNC homology 1 (BACH1) belongs to the Cap ‘n’ Collar (CNC) b-Zip family of proteins. There is no information about the role of BACH1 in regulating pulmonary vascular remodeling and PAH development. It was observed that BACH1 but not BACH2 expression was markedly increased in endothelial cells (ECs) in idiopathic PAH lungs compared to normal lungs, and in cultured human lung ECs treated with PDGF or hypoxia. Rats with endothelial cell-specific knockout of BACH1 via nanoparticle delivery of the CRISPR system exhibited markedly reduced pulmonary vascular remodeling and PAH in response to monocrotaline challenge. RNA sequencing analysis and in vivo rescue study have identified HIF-2a and PPARgamma as the downstream transcription factors responsible for BACH1-mediated pulmonary vascular remodeling and PAH. In vivo inhibition of PPARgamma and expression of HIf-2a mutant resistant to degradation together reversed the protective phenotype seen in BACH1 EC-specific knockout rats. Pharmacological treatment with BACH1 inhibitors ASP8731 and HPPE demonstrated marked reduction in right ventricular systolic pressure and RV hypertrophy in MCT-challenged rats, providing evidence that BACH1 inhibition effectively treats pulmonary hypertension. The data have for the first time demonstrated the fundamental role of endothelial BACH1 in mediating pulmonary vascular remodeling and PAH development. Based on the clinical relevance of these novel findings, inhibition of BACH1, especially endothelial BACH1 by small molecule inhibitor, siRNA, antisense, antibody, dominant negative BACH1, genome editing by CRISPR, Prime editing, Base editing, protein degrader, and other ways disrupting BACH1 expression or activity is a novel and effective PAH therapy.

As the molecular mechanisms responsible for pulmonary vascular remodeling remain elusive, there are limited options available for the prevention and treatment of progressive PAH⁴⁻⁶. Therapies targeting abnormalities in the prostacyclin, nitric oxide, and endothelin pathways result in only modest improvement in morbidity and mortality^{2,4-6}. The recent approved activin signaling inhibitor Sotatercept, although markedly reduces PA pressure and pulmonary vascular resistance, and improves right heart function, exhibits multicomponent improvement in only 39% PAH patients compared to 10% of placebo PAH patients⁷. PAH types with different etiologies share histopathologic features including obliterative intima thickening and complex plexiform lesions^{3,8}. It is fundamentally important to delineate the signaling pathway(s) mediating progressive pulmonary vascular remodeling, and thereby identify novel therapeutic strategies to inhibit or potentially reverse pulmonary vascular remodeling for PAH treatment.

The transcription factor BTB and CNC homology 1 (BACH1) comprised of 736 amino acids is a heme-binding protein belonging to the CNC-bZIP family^{9,10}. BACH1, ubiquitously expressed in many mammalian tissues, functions primarily as a transcriptional suppressor via heterodimerizing with small Maf proteins (e.g. Mafk) and binding to Maf recognition elements in the promoters of targeted genes at lower intracellular heme levels¹⁰⁻¹². Upon heme or cadimum binding or tyrosine phosphorylation (Y486) of BACH1, BACH1 is exported from the nucleus leading to upregulation of some target genes¹³⁻¹⁷. BACH1 plays an important role in regulating oxidative stresses and heme oxidation by transcriptional suppression of some oxidative stress-response genes, including heme oxygenase-1 (HO-1)¹¹ and NADPH quinone oxidoreductase 1(NQO1)¹⁸, by binding to Maf recognition elements in the gene promoters. It has been shown that BACH1 overexpression in endothelial cells (ECs) inhibits proliferation and induces apoptosis¹⁹. In smooth muscle cells (SMCs), loss of Bach1 inhibits SMC phenotypic switch from contractile to synthetic phenotype, and proliferation²⁰. Recent studies have shown that Bach1 suppresses ischemic angiogenesis in hindlimb ischemic injury via Wnt/p-catenin signaling²¹, and promotes neointima formation induced by wire injury through regulation of chromatin accessibility²⁰. Deletion of endothelial Bach1 attenuates atherosclerosis by reducing endothelial inflammation²². However, there is no report in the literature about the role of BACH1 in regulating pulmonary vascular remodeling and PAH development. Recent studies for the first time have defined the novel role of endothelial BACH1 in regulating pulmonary vascular remodeling and PAH development, delineated the underlying molecular mechanisms and provide novel therapeutic strategy to target endothelial BACH1 for PAH treatment.

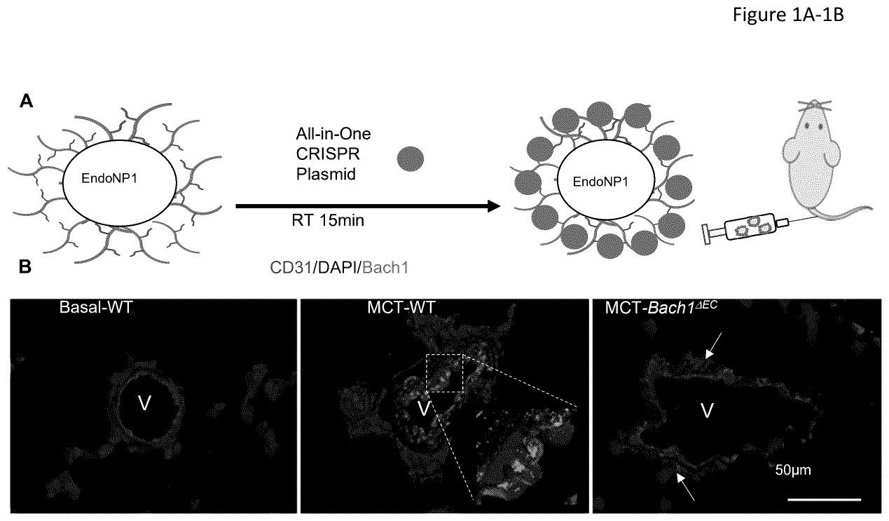

1. Generation of Bach1^{ΔEC} rats by endothelium-targeted nanoparticle delivery of the All-in-One CRISPR plasmid DNA. To generate a rat model with EC-specific disruption of Bach1 (Bach1^{ΔE}, i.e. CKO), the newly developed endothelium-targeted nanoparticle technology²³⁻²⁵ was employed to deliver the All-in-One CRISPR plasmid DNA expressing Cas9 under the control of the rat Cdh5 promoter and 2 rat Bach1-specific gRNAs (5′-GTCCGAGGATCGGAGTAGTG-3′ (SEQ ID NO: 1); 5′-ACGCACATGGACTGGCACGT-3′(SEQ ID NO: 2)) driven by U6 promoter (which exhibited >85% genome indel efficiency in in vitro screening) to rats at age of 4-5 weeks via tail vein injection (FIG. 1A). Plasmid DNA expressing scrambled gRNAs was delivered to WT rats as WT controls. At 8 weeks of age, the rats were challenged with MCT, lung tissues were collected at 4 weeks after MCT. Immunostaining shows diminished Bach1 protein expression in pulmonary vascular ECs of MCT-challenged Bach1^{ΔEC} rats whereas it was markedly induced in ECs of MCT-WT lungs (FIG. 1).

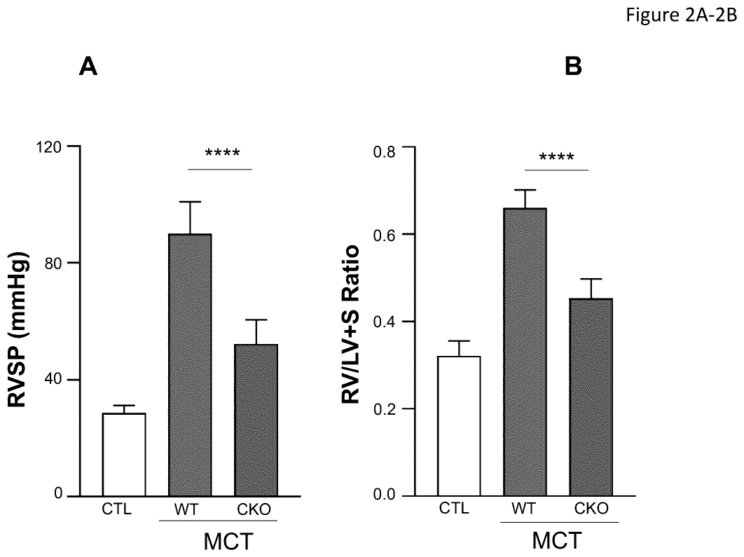

2. Disruption of endothelial Bach1 protects rats from monocrotaline (MCT)-induced PAH. To determine whether endothelial Bach1 is involved in the pathogenesis of PAH, the hemodynamics and cardiac function of Bach1^{ΔEC} (CKO) rats compared to WT rats (i.e. scrambled gRNA plasmid-delivered rats) at 4 weeks after MCT challenge were characterized. MCT-WT rats exhibited marked increases of right ventricular systolic pressure (RVSP) and RV/LV+S ratio indicative of hypertrophy (FIG. 2). However, RVSP and RV hypertrophy were markedly reduced in MCT-CKO rats, demonstrating that endothelial Bach1 deficiency inhibits MCT-induced PAH in rats.

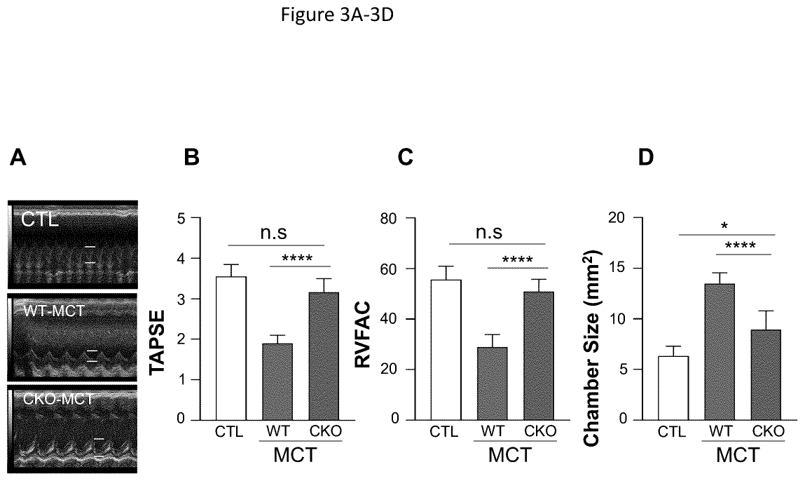

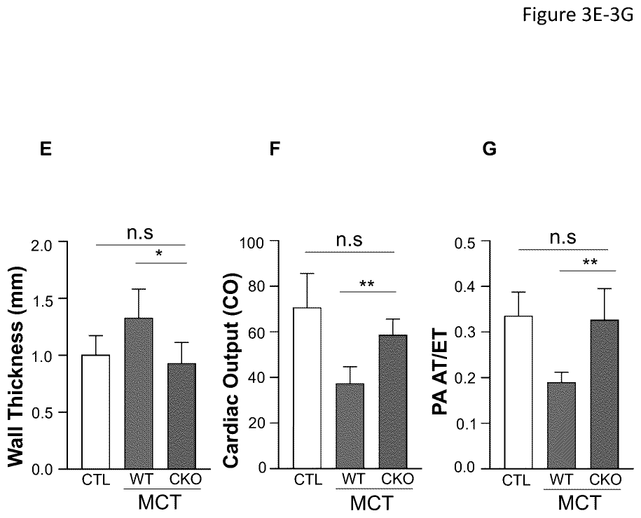

Echocardiography revealed marked decreases of TAPSE (RV function)²⁶, RVFAC (RV contractility), and cardiac output, and increased RV diastolic chamber size and RV wall thickness in MCT-WT rats whereas all these parameters except RV chamber size in MCT-CKO rats were in the levels similar to levels seen in control naive WT rats, demonstrating normal RV function in MCT-CKO rats (FIG. 3A-F). Pulmonary arterial (PA) function in MCT-CKO rats was also normal as the PAAT/ET ratio was similar to levels seen in naive WT rats whereas MCT-WT rats exhibited decreased PAAT/ET ratio indicating PA dysfunction (FIG. 3G).

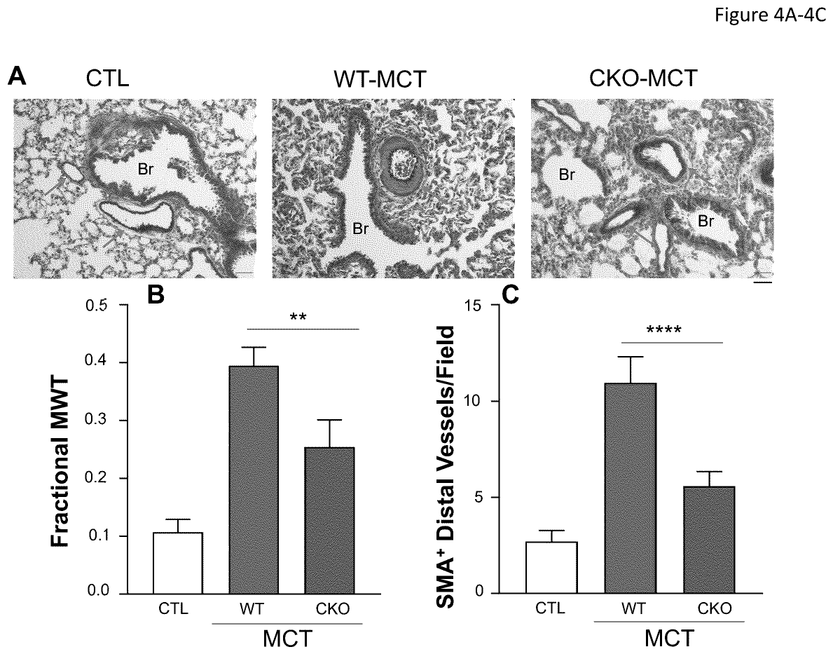

3. Inhibited pulmonary vascular remodeling in MCT-CKO rats. Next, pulmonary pathology examination by Russel-Movat pentachrome staining²⁷⁻²⁹, and anti-a-smooth muscle actin (SMA) immunostaining of these rat lung tissues was performed. MCT-induced increase of pulmonary vessel medial wall thickness in WT rats was attenuated in MCT-CKO rats (FIG. 4A, B). The number of muscularized distal pulmonary vessels was also markedly reduced in MCT-CKO rats compared to MCT-WT rats (FIG. 4C).

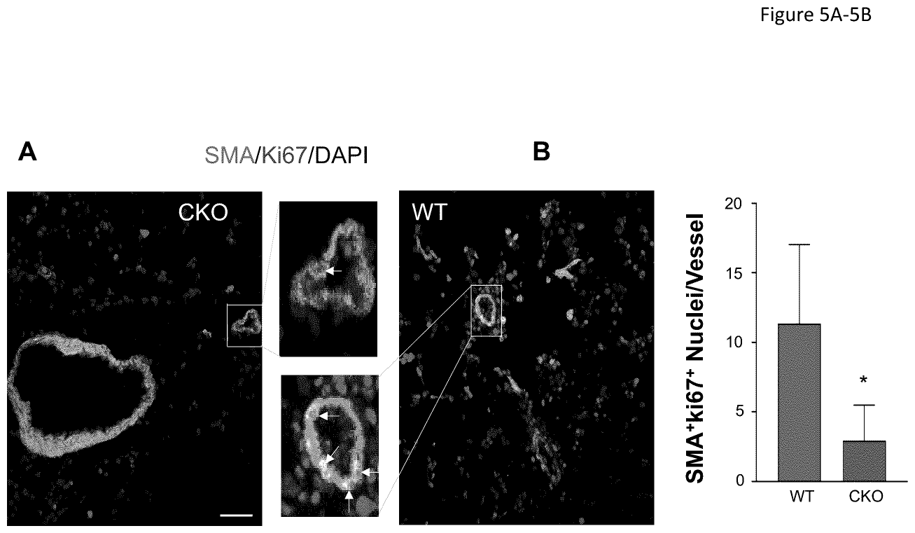

Pulmonary arterial smooth muscle cell (PASMC) proliferation was also assessed by immunostaining of the proliferating SMCs with Ki67. As shown in FIG. 5, PASMC proliferation in lungs of MCT-CKO rats was markedly reduced compared to that in WT-MCT rat lungs.

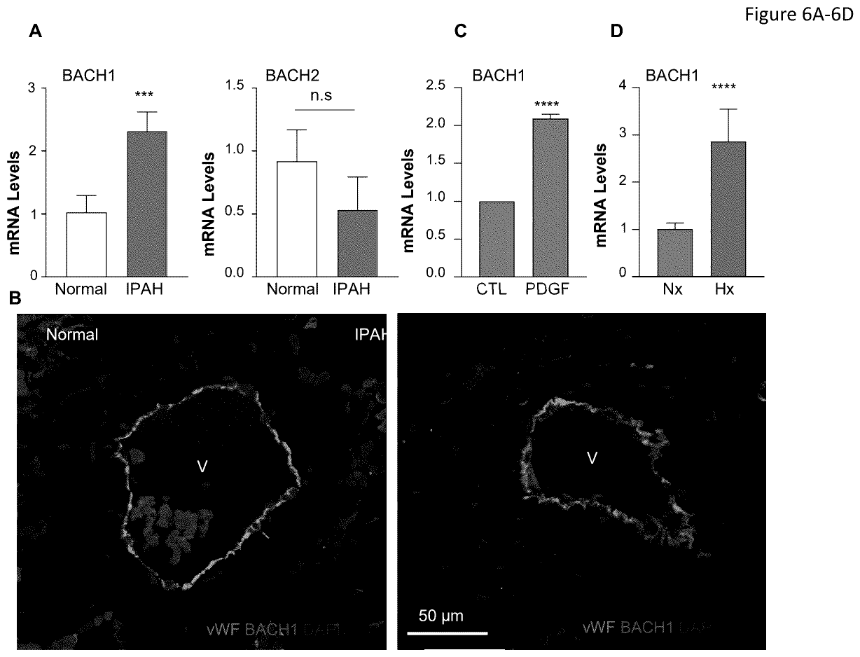

4. Marked increases of BACH1 expression in pulmonary vascular ECs of idiopathic PAH patients and in primary cultures of human pulmonary vascular ECs by PDGF treatment or in response to hypoxia. To address the clinical relevance of endothelial BACH1 in the pathogenesis of PAH, BACH1 expression in pulmonary vascular ECs of idiopathic PAH patients and healthy donors (normal) was assessed. Quantitative RT-PCR analysis revealed a marked increase of BACH1 but not BACH2 expression in primary cultures of human pulmonary arterial ECs isolated from healthy donors and idiopathic PAH patients (FIG. 6A). Immunostaining with anti-BACH1 and anti-vWF also showed marked increase of BACH1 protein expression in IPAH lung ECs (FIG. 6B).

It was next determined whether BACH1 expression in lung ECs was inducible by PAH-stimuli. Primary cultures of human pulmonary artery ECs from normal subjects were treated with PDGF and BACH1 expression was quantified by quantitative RT-PCR analysis. As shown in FIG. 6C, BACH1 was markedly induced by PDGF-BB treatment (50 ng/ml, 24 h). Similarly, BACH1 expression was markedly induced by hypoxia exposure (1% O₂, 24 h) in human lung microvascular ECs (FIG. 6D). BACH2 mRNA expression was not induced by either PDGF or hypoxia treatment (Data not shown). These data support the fundamental role of increased endothelial BACH1 expression in the pathogenesis of pulmonary vascular remodeling and PAH in patients.

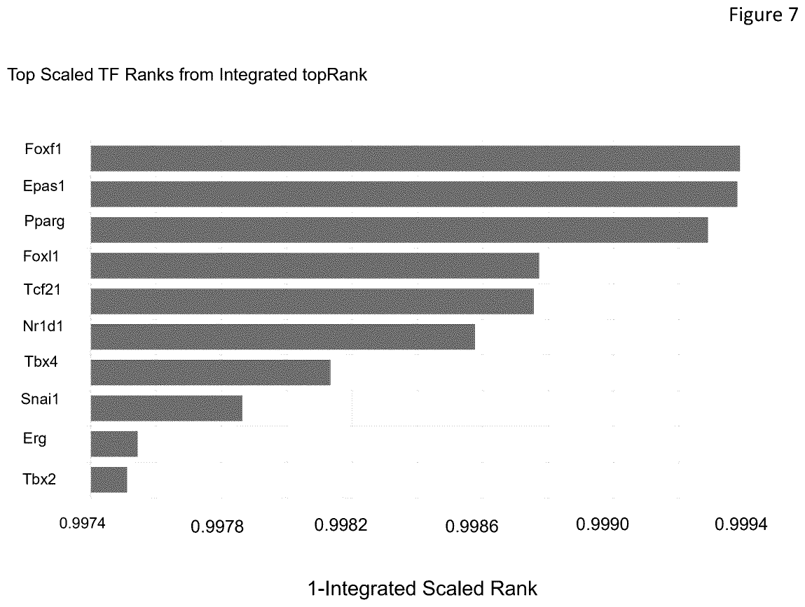

5. RNAseq analysis has identified HIF-2α and PPARγ as the top transcription factors regulating the expression of Differential Expression Genes (DEGs). To identify the pathways responsible for the protective phenotype in MCT-CKO rat lungs, RNAseq analysis of RNA samples from basal-WT, MCT-WT and MCT-CKO rat lungs was employed. There are 760 DEGs in MCT-WT and MCT-CKO lung samples. Transcription factor enrichment analysis has identified Foxf1, Epasl (i.e. Hif2a) and Pparg (i.e. PPARγ) as the top three transcription factors involved in the expression of the DEGs (FIG. 7). There are 30 DEGs regulated by Foxf1, 41 DEGs by HIF-2α and 56 DEGs by PPARγ. Taken together, HIF-2α and PPARγ are considered as the top 2 transcription factors.

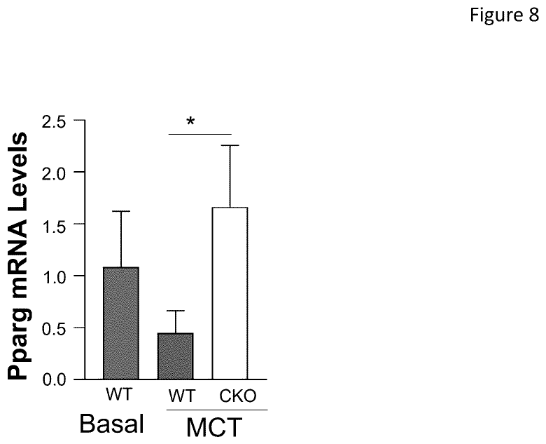

Quantitative RT-PCR analysis confirmed decreased Pparg expression in MCT-WT rat lungs compared to basal-WT rat lungs whereas Pparg expression was restored or even increased in MCT-CKO rat lungs (FIG. 8).

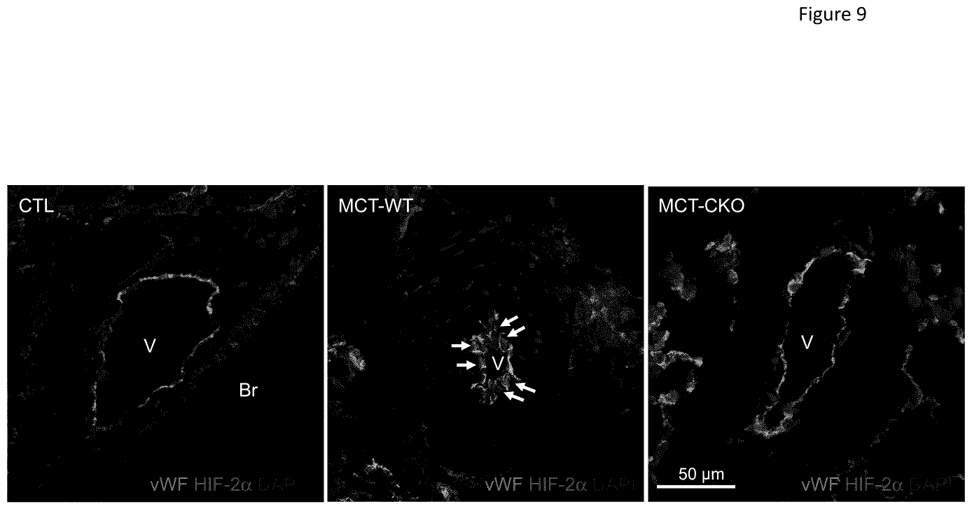

Immunostaining demonstrated prominent HIF-2α protein expression in pulmonary vascular ECs of MCT-WT rat lungs which was diminished in MCT-CKO rat lungs (FIG. 9). However, Quantitative RT-PCR analysis showed no difference in Hif2a mRNA expression (data not shown), consistent with the concept that HIF-2α expression is mainly regulated by post-translational modification and degradation not by transcriptional regulation.

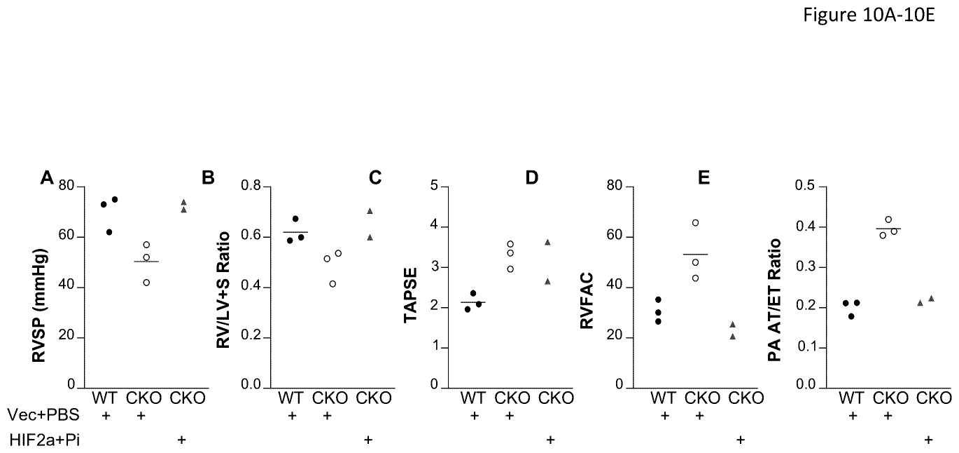

6. Combination treatment with endothelial expression of HIF-2α mutant and PPARγ inhibitor in CKO rats reversed the PAH phenotype seen in MCT-CKO rats. To explore the possibility of HIF-2α and PPARγ as downstream transcriptional factors of endothelial Bach1 regulating PAH development, EndoNP1 nanoparticles were employed to deliver plasmid DNA expressing HIF-2α mutant (Pro405Ala and Pro531Ala) resistant to ubiquitination-mediated proteasomal degradation^{30,31} under the control of rat Cdh5 promoter to ECs of CKO rats. SD rats at age of 4 weeks were administered with EndoNP1 nanoparticles:CRISPR plasmid DNA expressing Bach1-specifc gRNAs or scrambled gRNAs via tail-vein injection to generate CKO rats and control WT rats. At age of 6 weeks, these rats were challenged with MCT. Right after MCT challenge, the CKO rats were administered with the EndoNP1:HIF-2umutant plasmid DNA (20 μg/30 g BW, i.v. weekly) and PPARγ inhibitor GW9662 (1 mg/kg, i.p. daily)^{32,33}. WT and another group of CKO rats received EndoNP1:empty vector and PBS as controls. 3 weeks after MCT challenge, these rats were subjected to echocardiography and hemodynamic measurement. As shown in FIG. 10, RVSP was reduced in control MCT-CKO rats compared to control MCT-WT rats whereas completely reversed in the CKO rats with combination treatment. Reduced RV hypertrophy (RFV/LV+S ratio) in control MCT-CKO rats was also reversed in the treated CKO rats. Echocardiography demonstrated normalized RV function (FIG. 10C, D) and PA function (FIG. 10E). This rescue study demonstrates both HIF-2α stabilization/activation and PPARγ suppression in MCT-WT rats are responsible for endothelial BACH1-mediated PAH.

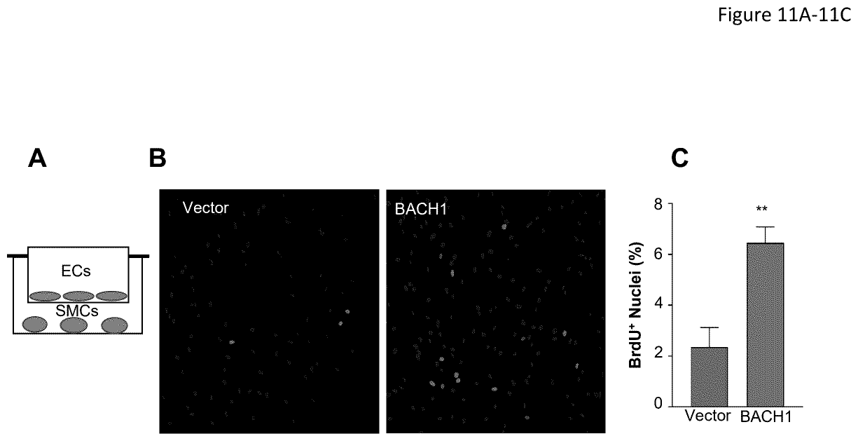

7. Endothelial cells overexpressing BACH1 induced human pulmonary arterial SMC proliferation in co-culture study. To determine if endothelial BACH1 can induce SMC hyperproliferation, an ECs and SMCs co-culture system was employed. Human lung microvascular ECs were transfected with plasmid DNA expressing BACH1 or empty vector. 24 h later, the cells were starved in serum-free SMC medium for 24 h and then placed on the top of the transwell system where PASMCs were grown at the bottom chamber with serum-free SMC medium for 24 h (FIG. 11A). 5-ethynyl 2′-deoxyuridine (Edu) was added to the bottom chamber for the last 4 h. 24 h after co-culture, PASMCs were fixed for immunostaining with anti-Edu antibody. As shown in FIG. 11, BACH1-overexpressing ECs induced a 3-fold increase of PASMC proliferation, demonstrating the important role of upregulated BACH1 expression in ECs by PAH-causing factors in promoting pulmonary vascular remodeling.

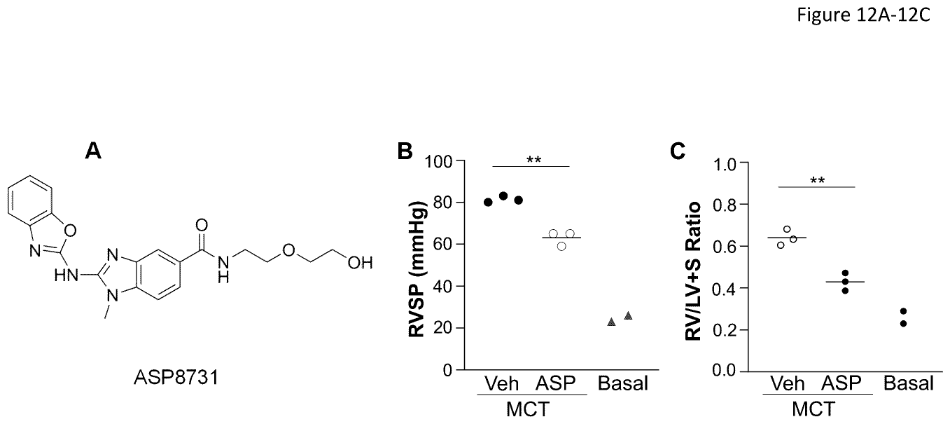

8. Pharmacological treatment with BACH1 inhibitors inhibited PH in MCT-challenged rats. To determine whether BACH1 inhibitor treatment can inhibit PH, MCT rats were treated with BACH1 inhibitors. First, the effects of BACH1 inhibitor ASP8731 (34, 35) were tested. At 14 days after MCT challenge, the rats were treated with either vehicle (PBS) or ASP8731 (25 mg/kg, gavage, daily). At 28 days post-MCT challenge, the rats were subject to hemodynamic measurement. As shown in FIG. 12, ASP8731 treatment led to marked reduction of RVSP. Furthermore, RV/LV+S ratio was also markedly reduced in ASP8731-treated rats compared to vehicle-treated rats, indicative of reduced RV hypertrophy.

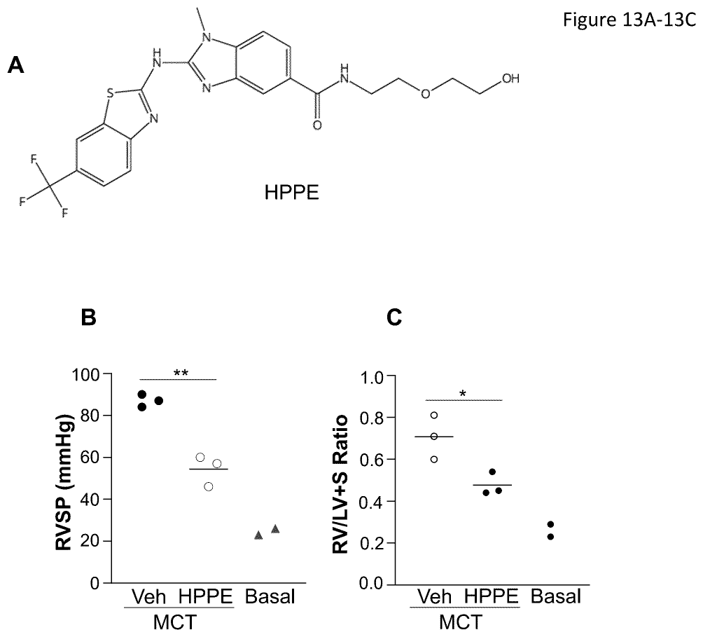

The effects of another BACH1 inhibitor HPPE (36) were also assessed. At 10 days post-MCT challenge, the rats were treated with either vehicle or HPPE (50 mg/kg, daily, i.p., every 4 days with 1 day interval). At 28 days post-MCT challenge, RVSP was measured and RV hypertrophy was assessed. HPPE treatment markedly inhibited RVSP and RV/LV+S ratio (FIG. 13).

Together, these data provide evidence that BACH1 inhibition by BACH1 inhibitor treatment inhibited PH in a rat PH model.

Conclusion. In summary, it has been established that endothelial BACH1 mediates pulmonary vascular remodeling and PAH development, and novel mechanisms of endothelial BACH1 regulation of pulmonary vascular remodeling by both transcriptional suppression of PPARγ and HIF-2α protein stabilization in ECs have been delineated. Prominent BACH1 expression in pulmonary vascular ECs of idiopathic PAH patients was also observed and BACH1 was markedly induced in primary culture of human lung ECs by PAH-causing stimuli. Pharmacological treatment with BACH1 inhibitors ASP8731 or HPPE demonstrated marked reduction in right ventricular systolic pressure and RV hypertrophy in MCT-challenged rats, providing evidence that BACH1 inhibition effectively treats pulmonary hypertension. Thus, targeting endothelial BACH1 is a novel effective therapeutic approach for PAH treatment.

### REFERENCE

1. V. V. McLaughlin et al., ACCF/AHA 2009 expert consensus document on pulmonary hypertension: a report of the American College of Cardiology Foundation Task Force on Expert Consensus Documents and the American Heart Association: developed in collaboration with the American College of Chest Physicians, American Thoracic Society, Inc., and the Pulmonary Hypertension Association. Circulation 119, 2250-2294 (2009).
2. H. W. Farber, The status of pulmonary arterial hypertension in 2008. Circulation 117, 2966-2968 (2008).
3. E. Stacher, B. B. Graham, J. M. Hunt, A. Gandjeva, S. D. Groshong, V. V. McLaughlin, M. Jessup, W. E. Grizzle, M. A. Aldred, C. D. Cool, R. M. Tuder, Modern age pathology of pulmonary arterial hypertension. American journal of respiratory and critical care medicine 186, 261-272 (2012).
4. G. Sutendra, E. D. Michelakis, Pulmonary arterial hypertension: challenges in translational research and a vision for change. Science translational medicine 5, 208sr205 (2013).
5. D. B. Taichman, J. Ornelas, L. Chung, J. R. Klinger, S. Lewis, J. Mandel, H. I. Palevsky, S. Rich, N. Sood, E. B. Rosenzweig, T. K. Trow, R. Yung, C. G. Elliott, D. B. Badesch, Pharmacologic therapy for pulmonary arterial hypertension in adults: CHEST guideline and expert panel report. Chest 146, 449-475 (2014).
6. V. V. McLaughlin, S. J. Shah, R. Souza, M. Humbert, Management of pulmonary arterial hypertension. Journal of the American College of Cardiology 65, 1976-1997 (2015).
7. M. M. Hoeper et al., Phase 3 Trial of Sotatercept for Treatment of Pulmonary Arterial Hypertension. The New England journal of medicine 388, 1478-1490 (2023).
8. M. Humbert, C. Guignabert, S. Bonnet, P. Dorfmuller, J. R. Klinger, M. R. Nicolls, A. J. Olschewski, S. S. Pullamsetti, R. T. Schermuly, K. R. Stenmark, M. Rabinovitch, Pathology and pathobiology of pulmonary hypertension: state of the art and research perspectives. The European respiratory journal 53, (2019).
9. J. L. Blouin, G. Duriaux Sail, M. Guipponi, C. Rossier, M. P. Pappasavas, S. E. Antonarakis, Isolation of the human BACH1 transcription regulator gene, which maps to chromosome 21q22.1. Human genetics 102, 282-288 (1998).
10. T. Oyake, K. Itoh, H. Motohashi, N. Hayashi, H. Hoshino, M. Nishizawa, M. Yamamoto, K. Igarashi, Bach proteins belong to a novel family of BTB-basic leucine zipper transcription factors that interact with MafK and regulate transcription through the NF-E2 site. Molecular and cellular biology 16, 6083-6095 (1996).
11. J. Sun, H. Hoshino, K. Takaku, O. Nakajima, A. Muto, H. Suzuki, S. Tashiro, S. Takahashi, S. Shibahara, J. Alam, M. M. Taketo, M. Yamamoto, K. Igarashi, Hemoprotein Bach1 regulates enhancer availability of heme oxygenase-1 gene. The EMBO journal 21, 5216-5224 (2002).
12. S. Dhakshinamoorthy, A. K. Jain, D. A. Bloom, A. K. Jaiswal, Bach1 competes with Nrf2 leading to negative regulation of the antioxidant response element (ARE)-mediated NAD(P)H:quinone oxidoreductase 1 gene expression and induction in response to antioxidants. The Journal of biological chemistry 280, 16891-16900 (2005).
13. K. Ogawa, J. Sun, S. Taketani, O. Nakajima, C. Nishitani, S. Sassa, N. Hayashi, M. Yamamoto, S. Shibahara, H. Fujita, K. Igarashi, Heme mediates derepression of Maf recognition element through direct binding to transcription repressor Bach1. The EMBO journal 20, 2835-2843 (2001).
14. Y. Zenke-Kawasaki, Y. Dohi, Y. Katoh, T. Ikura, M. Ikura, T. Asahara, F. Tokunaga, K. Iwai, K. Igarashi, Heme induces ubiquitination and degradation of the transcription factor Bach1. Molecular and cellular biology 27, 6962-6971 (2007).
15. J. W. Kaspar, A. K. Jaiswal, Antioxidant-induced phosphorylation of tyrosine 486 leads to rapid nuclear export of Bach1 that allows Nrf2 to bind to the antioxidant response element and activate defensive gene expression. The Journal of biological chemistry 285, 153-162 (2010).
16. H. Suzuki, S. Tashiro, J. Sun, H. Doi, S. Satomi, K. Igarashi, Cadmium induces nuclear export of Bach1, a transcriptional repressor of heme oxygenase-1 gene. The Journal of biological chemistry 278, 49246-49253 (2003).
17. H. Suzuki, S. Tashiro, S. Hira, J. Sun, C. Yamazaki, Y. Zenke, M. Ikeda-Saito, M. Yoshida, K. Igarashi, Heme regulates gene expression by triggering Crm1-dependent nuclear export of Bach1. The EMBO journal 23, 2544-2553 (2004).
18. S. Dhakshinamoorthy, A. K. Jain, D. A. Bloom, A. K. Jaiswal, Bach1 competes with Nrf2 leading to negative regulation of the antioxidant response element (ARE)-mediated NAD(P)H:quinone oxidoreductase 1 gene expression and induction in response to antioxidants. The Journal of biological chemistry 280, 16891-16900 (2005).
19. X. Wang, J. Liu, L. Jiang, X. Wei, C. Niu, R. Wang, J. Zhang, D. Meng, K. Yao, Bach1 Induces Endothelial Cell Apoptosis and Cell-Cycle Arrest through ROS Generation. Oxidative medicine and cellular longevity 2016, U.S. Pat. No. 6,234,043 (2016).
20. J. Guo, J. Qiu, M. Jia, Q. Li, X. Wei, L. Li, Q. Pan, J. Jin, F. Ge, S. Ma, Y. He, J. Lin, Y. Li, J. Ma, N. Jiang, X. Zhi, L. Jiang, J. Zhang, E. Osto, Q. Jing, X. Wang, D. Meng, BACH1 deficiency prevents neointima formation and maintains the differentiated phenotype of vascular smooth muscle cells by regulating chromatin accessibility. Nucleic acids research 51, 4284-4301 (2023).
21. L. Jiang, M. Yin, X. Wei, J. Liu, X. Wang, C. Niu, X. Kang, J. Xu, Z. Zhou, S. Sun, X. Wang, X. Zheng, S. Duan, K. Yao, R. Qian, N. Sun, A. Chen, R. Wang, J. Zhang, S. Chen, D. Meng, Bach1 Represses Wnt/β-Catenin Signaling and Angiogenesis. Circulation research 117, 364-375 (2015).
22. M. Jia, Q. Li, J. Guo, W. Shi, L. Zhu, Y. Huang, Y. Li, L. Wang, S. Ma, T. Zhuang, X. Wang, Q. Pan, X. Wei, Y. Qin, X. Li, J. Jin, X. Zhi, J. Tang, Q. Jing, S. Li, L. Jiang, L. Qu, E. Osto, J. Zhang, X. Wang, B. Yu, D. Meng, Deletion of BACH1 Attenuates Atherosclerosis by Reducing Endothelial Inflammation. Circulation research 130, 1038-1055 (2022).
23. X. Zhang, H. Jin, X. Huang, B. Chaurasiya, D. Dong, T. P. Shanley, Y. Y. Zhao, Robust genome editing in adult vascular endothelium by nanoparticle delivery of CRISPR-Cas9 plasmid DNA. Cell reports 38, 110196 (2022).
24. B. Liu, Y. Peng, D. Yi, N. Machireddy, D. Dong, K. Ramirez, J. Dai, R. Vanderpool, M. M. Zhu, Z. Dai, Y. Y. Zhao, Endothelial PHD2 deficiency induces nitrative stress via suppression of caveolin-1 in pulmonary hypertension. The European respiratory journal 60, (2022).
25. X. Huang, X. Zhang, N. Machireddy, C. E. Evans, S. D. Trewartha, G. Hu, Y. Fang, G. M. Mutlu, D. Wu, Y. Y. Zhao, Endothelial FoxMl reactivates aging-impaired endothelial regeneration for vascular repair and resolution of inflammatory lung injury. Science translational medicine 15, eabm5755 (2023).
26. S. Ghio, F. Recusani, C. Klersy, R. Sebastian, M.L. Laudisa, C. Campana, A. Gavazzi, L. Tavazzi. Prognostic usefulness of the tricuspid annular plane systolic excursion in patients with congestive heart failure secondary to idiopathic or ischemic dilated cardiomyopathy. American Journal of Cardiology 85, 837-42 (2000).
27. Z. Dai, M. Li, J. Wharton, M. M. Zhu, Y. Y. Zhao, Prolyl-4 Hydroxylase 2 (PHD2) Deficiency in Endothelial Cells and Hematopoietic Cells Induces Obliterative Vascular Remodeling and Severe Pulmonary Arterial Hypertension in Mice and Humans Through Hypoxia-Inducible Factor-2a. Circulation 133, 2447-2458 (2016).
28. Z. Dai, et al. Therapeutic Targeting of Vascular Remodeling and Right Heart Failure in Pulmonary Arterial Hypertension with a HIF-2α Inhibitor. American journal of respiratory and critical care medicine 198, 1423-1434 (2018).
29. Russell HK, Jr. A modification of Movat's pentachrome stain. Arch Pathol. 94, 187-91(1972).
30. M.M. Zhu, J. Dai, Z. Dai, Y. Peng, Y.Y. Zhao. GCN2 kinase acitvation mediates pulmonary vascular remodeling and pulmonary arterial hypertension. JCI Insight. 9, e177926 (2024).
31. S.V. Mendoza, et al. Degradation-Resistant Hypoxia Inducible Factor-2a in Murine Osteocytes Promotes a High Bone Mass Phenotype. The Journal of Bone and Mineral Research Plus 7, e10724 (2023).
32. A. Baumann, et al., GW9662, a peroxisome proliferator-activated receptor gamma antagonist, attenuates the development of non-alcoholic fatty liver disease. Metabolism: clinical and experimental 133, 155233 (2022).
33. M. Collino, et al. The selective PPARgamma antagonist GW9662 reverses the protection of LPS in a model of renal ischemia-reperfusion. Kidney international 68, 529-536 (2005).
34. J. D. Belcher, et al. The BACH1 inhibiotr ASP8731 inhibits inflammation and vaso-occlusion and induces fetal hemoglobin in sickle cell disease. Frontier in Medicine 10, 1101501 (2023).
35. Nataraja S, et al. ML-0207/ASP8731: a novel BACH1 inhibitor that induces fetal hemoglobin in treatment of sickle cell disease. Blood. 2021; 138(Supplement 1):854-54.
36. Ahuja M, et al. Bach1 derepression is neuroprotective in a mouse model of Parkinson's disease. Proc Natl Acad Sci USA. 2021 Nov. 9; 118(45):e21 11643118.

What is claimed:

1. A method of treating pulmonary hypertension in a subject in need thereof, the method comprising administering an inhibitor of BACH1 to the subject.

2. The method of claim 1, wherein pulmonary hypertension is pulmonary arterial hypertension.

3. The method of claim 1, wherein the BACH1 inhibitor is selected from the group consisting of a small molecule therapeutic agent, an antibody, a peptide, a miRNA, a siRNA, an oligonucleotide, a genome editor, a protein degrader, a cytokine, an agonist, and any combination thereof.

4. The method of claim 3, wherein the BACH1 inhibitor is selected from the group consisting of ASP8731, HPP-4382, HPP971, HPPA, HPPB, HPPC, HPPD, HPPE, TBE56, TBE53, TBE31, cannabiquinone, cannabidiol, IMM004, hemin, CDDO-TFEA, CDDO-Me, BI033, and analogs thereof, and any combination thereof.

5. The method of claim 1, wherein the BACH1 inhibitor decreases vascular resistance.

6. The method of claim 5 wherein the vascular resistance is pulmonary vascular resistance.

7. The method of claim 1, wherein the BACH1 inhibitor decreases vascular remodeling.

8. The method of claim 7, wherein the vascular remodeling is pulmonary vascular remodeling.

9. The method of claim 1, wherein the BACH1 inhibitor decreases BACH1 function and/or expression in endothelial cells.

10. A method of inhibiting or reducing vascular remodeling in a subject in need, the method comprising administering a BACH1 inhibitor to the subject.

11. The method of claim 10, wherein the BACH1 inhibitor is selected from the group consisting of a small molecule therapeutic agent, an antibody, a peptide, a miRNA, a siRNA, an oligonucleotide, a genome editor, a protein degrader, a cytokine, an agonist, and any combination thereof.

12. The method of claim 11, wherein the BACH1 inhibitor is selected from the group consisting of ASP8731, HPP-4382, HPP971, HPPA, HPPB, HPPC, HPPD, HPPE, TBE31, TBE53, TBE56, cannabiquinone, cannabidiol, IMM004, Hemin, CDDO-TFEA, CDDO-Me, BI033, and analogs thereof, and any combination thereof.

13. The method of claim 10, wherein the vascular remodeling comprises pulmonary vascular remodeling.

14. The method of claim 10, wherein the method decreases vascular resistance.

15. The method of claim 10, wherein the BACH1 inhibitor decreases BACH1 function and/or expression in endothelial cells.

16. A method of inhibiting or reducing pulmonary vascular remodeling in a subject in need, the method comprising administering an inhibitor of BACH1 to the subject, wherein the subject is diagnosed with or suspected of having pulmonary hypertension.

17. The method of claim 16, wherein the BACH1 inhibitor is selected from the group consisting of a small molecule therapeutic agent, an antibody, a peptide, a miRNA, a siRNA, an oligonucleotide, a genome editor, a protein degrader, a cytokine, an agonist, and any combination thereof.

18. The method of claim 17, wherein the BACH1 inhibitor is selected from the group consisting of ASP8731, HPP-4382, HPP971, HPPA, HPPB, HPPC, HPPD, HPPE, TBE31, TBE53, TBE56, cannabiquinone, cannabidiol, IMM004, Hemin, CDDO-TFEA, CDDO-Me, BI033, and analogs thereof, and any combination thereof.

19. The method of claim 17, wherein the method decreases vascular resistance.


SEQUENCE LISTING (2 sequences)

SEQ ID NO: 1 - DNA, 20 bp, synthetic construct
  source 1..20: mol_type = other DNA
gtccgaggatcggagtagtg

SEQ ID NO: 2 - DNA, 20 bp, synthetic construct
  source 1..20: mol_type = other DNA
acgcacatggactggcacgt